(19) United States
(12) Patent Application Publication
(10) Pub. No.: US 2024/0350170 A1
(43) Pub. Date: Oct. 24, 2024

(54) COILED WIRE ELECTRODE

(71) Applicant: Boston Scientific Medical Device Limited, Galway (IE)

(72) Inventor: Lauren Koon, Mississauga (CA)

(21) Appl. No.: 18/637,185

(22) Filed: Apr. 16, 2024

Related U.S. Application Data
(60) Provisional application No. 63/497,158, filed on Apr. 19, 2023.

(51) Int. Cl. A61B 17/34; A61B 5/283; A61B 5/367; A61B 17/00

(52) U.S. Cl. CPC A61B 17/3476; A61B 5/283; A61B 5/367; A61B 2017/00526; A61B 2562/125

(57) ABSTRACT

A medical device includes an elongate body including a proximal portion and a distal portion. The device includes an electrode located on the distal portion. A conductor extends from the electrode to the proximal portion. The electrode and the conductor are formed of a single piece of continuous material.

### CROSS REFERENCE TO RELATED APPLICATIONS

The present application claims the benefit of U.S. Provisional Application No. 63/497,158 entitled “COILED WIRE ELECTRODE,” filed Apr. 19, 2023, which is hereby incorporated by reference in its entirety.

### TECHNICAL FIELD

The present invention relates generally to methods and devices usable within the body of a patient. More specifically, the present invention is concerned with an apparatus pertaining to a medical device, such as a sheath, dilator, piercing device, or catheter requiring an electrode.

### BACKGROUND

In manufacturing medical devices having small electrodes there can be many challenges. Typically, electrodes are formed as solid metal bands that are welded or soldered to a conductor. Commonly, stainless steel or platinum-iridium are used. Welding dissimilar materials can create a joint that may have variability in strength for mechanical purposes. For example, a flat weld might break or the electrode and attached conductor might break and disconnect. Alternatively, if the conductor and electrode are not welded flat, it may be challenging to create a connection that is flush, has a constant thickness, or has a small enough thickness to assemble with a thin-walled device.

### SUMMARY

Example 1 is a medical device having an elongate body including a proximal portion and a distal portion. The device includes an electrode located on the distal portion. A conductor extends from the electrode to the proximal portion. The electrode and the conductor are formed of a single piece of continuous material.

Example 2 is the medical device of Example 1 wherein the elongate body includes a lumen extending from the proximal portion to the distal portion.

Example 3 is the medical device of Example 1 or 2 wherein the medical device is a dilator, guidewire, catheter, or perforation device.

Example 4 is the medical device of any of Examples 1 to 3 wherein the device includes a first surface electrode.

Example 5 is the medical device of Example 4 wherein the first surface electrode is positioned proximal of the electrode.

Example 6 is the medical device of Example 4 wherein the electrode and the first surface electrode have a same diameter.

Example 7 is the medical device of Example 4 wherein the electrode and the first surface electrode have different lengths.

Example 8 is the medical device of any of Examples 1 to 7 wherein the electrode comprises a plurality of loops positioned along a longitudinal axis of the elongate body

Example 9 is the medical device of any of Examples 1 to 7 wherein the electrode includes a plurality of undulations.

Example 10 is the medical device of Example 9 wherein the plurality of undulations are arranged parallel or perpendicular to a longitudinal axis of the elongated body.

Example 11 is the medical device of any of Examples 8 to 10 wherein the plurality of loops or plurality of undulations are joined by a conductive material.

Example 12 is the medical device of any of Examples 1 to 11 wherein the conductor extends along a lumen to the proximal end.

Example 13 is the medical device of any of Examples 1 to 11 wherein the conductor is encapsulated in a wall of the elongate body.

Example 14 is the medical device of any of Examples 1 to 11 wherein the conductor extends along a surface of the elongate body.

Example 15 is the medical device of any of Examples 1 to 14 wherein the distal portion includes a taper.

Example 16 is a medical device including an elongate body having a proximal portion, a distal portion, and a lumen extending from the proximal portion to the distal portion. The device includes an electrode located on the distal portion. A conductor extends from the electrode to the proximal portion. The electrode and the conductor are formed of a single piece of continuous material.

Example 17 is the medical device of Example 16 wherein the medical device is a dilator, guidewire, catheter, or perforation device.

Example 18 is the medical device of Example 16 wherein the device includes a first surface electrode is positioned proximal of the electrode.

Example 19 is the medical device of Example 18 wherein the device includes a second surface electrode is positioned proximal of the first surface electrode.

Example 20 is the medical device of Example 19 wherein, the electrode, the first surface electrode, and the second surface electrode have a same diameter.

Example 21 is the medical device of Example 19 wherein the first surface electrode and the second surface electrode have different lengths.

Example 22 is the medical device of Example 16 wherein the electrode comprises a plurality of loops positioned along a longitudinal axis of the elongate body.

Example 23 is the medical device of Example 16 wherein the electrode includes a plurality of undulations.

Example 24 is the medical device of Example 23 wherein the plurality of undulations are arranged parallel or perpendicular to a longitudinal axis of the elongated body.

Example 25 is the medical device of Example 22 wherein the plurality of loops are joined by a conductive material.

Example 26 is the medical device of Example 16 wherein the conductor extends within the lumen to the proximal end.

Example 27 is the medical device of Example 16 wherein the conductor is encapsulated in a wall of the elongate body.

Example 28 is the medical device of Example 16 wherein the conductor extends along a surface of the elongate body.

Example 29 is the medical device of Example 16 wherein the distal portion includes a taper.

Example 30 is a medical device having an elongate body including a proximal portion and a tapered distal end. A coiled electrode is located on the tapered distal end.

A conductor extends from the coiled electrode to the proximal portion. The coiled electrode and the conductor are formed of a single piece of continuous material.

Example 31 is the medical device of Example 30 wherein a first surface electrode is positioned proximal of the coiled electrode.

Example 32 is the medical device of Example 31 wherein a second surface electrode positioned proximal of the first surface electrode.

Example 33 is the medical device of Example 32 wherein the coiled electrode, the first surface electrode, and the second surface electrode have different lengths.

Example 34 is the medical device of Example 30 wherein the coiled electrode comprises a plurality of loops positioned along a longitudinal axis of the elongate body.

Example 35 is a method for fabricating a medical device. The method includes forming a coiled electrode by wrapping a continuous wire around a template for a plurality of turns. The method includes placing the coiled electrode on a distal portion of the medical device. The method also includes threading a proximal portion of the continuous wire though the medical device to a proximal end of the medical device.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIGS. 1A-1C are schematic illustrations of a medical procedure within a patient's heart utilizing a transseptal access system according to embodiments of the disclosure.

FIG. 2 illustrates a dilator in accordance with an embodiment of the present disclosure.

FIG. 3 illustrates a medical device in accordance with an embodiment of the present disclosure.

FIGS. 4A-4C illustrate various configurations for a coiled electrode in accordance with the present disclosure.

FIG. 5 illustrates an embodiment for joining coils of a coiled electrode in accordance with the present disclosure.

FIG. 6 illustrates a medical device in accordance with an embodiment of the present disclosure.

FIGS. 7A-7D illustrate cross-sections of wire used for forming coiled electrodes in accordance with the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

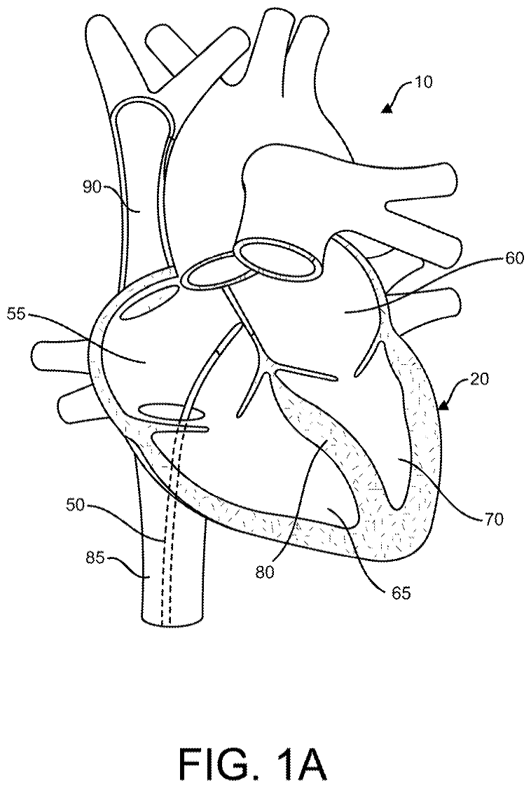

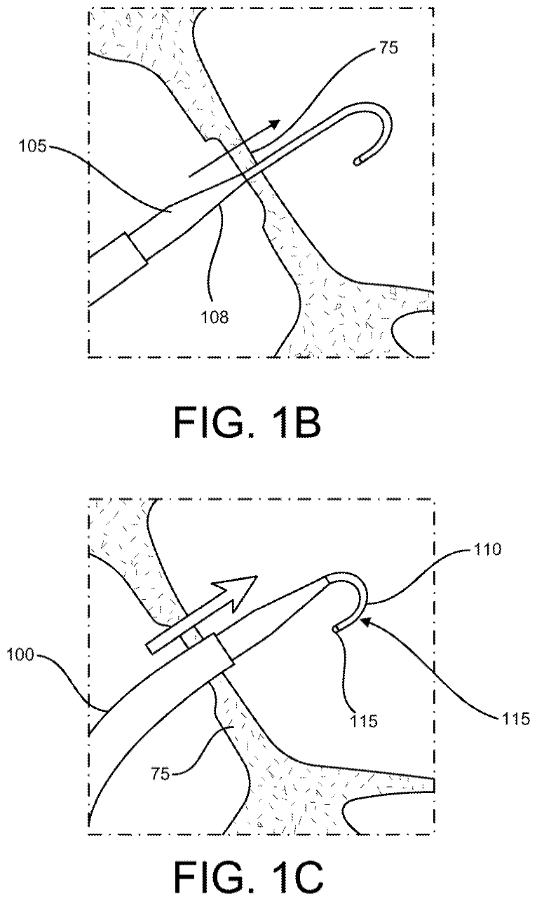

FIGS. 1A-1C are schematic illustrations of a medical procedure 10 within a patient's heart 20 utilizing a transseptal access system 50 according to embodiments of the disclosure. As is known, the human heart 20 has four chambers, a right atrium 55, a left atrium 60, a right ventricle 65 and a left ventricle 70. Separating the right atrium 55 and the left atrium 60 is an atrial septum 75 and separating the right ventricle 65 and the left ventricle 70 is a ventricular septum 80. As is further known, deoxygenated blood from the patient's body is returned to the right atrium 55 via an inferior vena cava (IVC) 85 or a superior vena cava (SVC) 90.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

The medical procedure 10 illustrated in FIGS. 1A-1C is an exemplary embodiment for providing access to the left atrium 60 using the transseptal access system 50 for subsequent deployment of the aforementioned diagnostic and/or therapeutic devices within the left atrium 60. As shown in FIGS. 1A-1C, target tissue site can be defined by tissue on the atrial septum 75. In the illustrated embodiment, the target site is accessed via the IVC 85, for example through the femoral vein, according to conventional catheterization techniques. In other embodiments, access to the target site on the atrial septum 75 may be accomplished using a superior approach wherein the transseptal access system 50 is advanced into the right atrium 55 via the SVC 90.

In the illustrated embodiment, the transseptal access system 50 includes an introducer sheath 100, a dilator 105 having a dilator body 107 and a tapered distal tip portion 108, and a radiofrequency (RF) perforation device 110, also known as a piercing device, having distal end portion 112 terminating in a tip electrode 115. As shown, in the assembled use state illustrated in FIGS. 1A-1C, the RF perforation device 110 can be disposed within the dilator 105, which itself can be disposed within the sheath 100. In one embodiment in which the transseptal access system 50 is deployed into the right atrium 55 via the IVC 105, a user introduces a guidewire (not shown) into a femoral vein, typically the right femoral vein, and advances it towards the heart 20. The sheath 100 may then be introduced into the femoral vein over the guidewire, and advanced towards the heart 20. In one embodiment, the distal ends of the guidewire and sheath 100 are then positioned in the SVC 90. These steps may be performed with the aid of an imaging system, e.g., fluoroscopy or ultrasonic imaging. The dilator 105 may then be introduced into the sheath 100 and over the guidewire, and advanced through the sheath 100 into the SVC 90. Alternatively, the dilator 105 may be fully inserted into the sheath 100 prior to entering the body, and both may be advanced simultaneously towards the heart 20. When the guidewire, sheath 100, and dilator 105 have been positioned in the superior vena cava, the guidewire is removed from the body, and the sheath 100 and the dilator 105 are retracted so that their distal ends are positioned in the right atrium 55. The RF perforation device 110 described can then be introduced into the dilator 105, and advanced toward the heart 20.

Subsequently, the user may position the distal end of the dilator 105 against the atrial septum 75, which can be done under imaging guidance. The RF perforation device 110 is then positioned such that electrode 115 is aligned with or protruding slightly from the distal end of the dilator 105. The dilator 105 and the RF perforation device 110 may be dragged along the atrial septum 75 and positioned, for example against the fossa ovalis of the atrial septum 75 under imaging guidance. A variety of additional steps may be performed, such as measuring one or more properties of the target site, for example an electrogram or ECG (electrocardiogram) tracing and/or a pressure measurement, or delivering material to the target site, for example delivering a contrast agent. Such steps may facilitate the localization of the tip electrode 115 at the desired target site. In addition, tactile feedback provided by medical RF perforation device 110 is usable to facilitate positioning of the tip electrode 115 at the desired target site.

With the tip electrode 115 and dilator 105 positioned at the target site, energy is delivered from an energy source, e.g., an RF generator, through the RF perforation apparatus 110 to the tip electrode 115 and the target site. In some embodiments, the energy is delivered at a power of at least about 5 W at a voltage of at least about 75 V (peak-to-peak), and functions to vaporize cells in the vicinity of the tip electrode 115, thereby creating a void or perforation through the tissue at the target site. The user then applies force to the RF perforation device 110 so as to advance the tip electrode 115 at least partially through the perforation. In these embodiments, when the tip electrode 115 has passed through the target tissue, that is, when it has reached the left atrium 60, energy delivery is stopped. In some embodiments, the step of delivering energy occurs over a period of between about 1 s and about 5 s.

With the tip electrode 115 of the RF perforation device 110 having crossed the atrial septum 75, the dilator 105 can be advanced forward, with the tapered distal tip portion 107 operating to gradually enlarge the perforation to permit advancement of the distal end of the sheath 100 into the left atrium 60.

In some embodiments, the distal end portion 112 of the RF perforation device 110 may be pre-formed to assume an atraumatic shape such as a J-shape (as shown in FIGS. 1B-1C), a pigtail shape or other shape selected to direct the tip electrode 115 away from the endocardial surfaces of the left atrium 60. Examples of such RF perforation devices can be found, for example, in U.S. patent application Ser. Nos. 16/445,790 and 16/346,404 assigned to Baylis Medical Company, Inc. The aforementioned pre-formed shapes can advantageously function to minimize the risk of unintended contact between the tip electrode 115 and tissue within the left atrium 60 and can also operate to anchor the distal end portion 112 within the left atrium 60 during subsequent procedural steps. For example, in embodiments, the RF perforation device 110 can be structurally configured to function as a delivery rail for deployment of a relatively larger bore therapy delivery sheath and associated dilator(s). In such embodiments, the dilator 105 and the sheath 100 are withdrawn following deployment of the distal end portion 112 of the RF perforation device 110 into the left atrium 60. The anchoring function of the pre-formed distal end portion 112 inhibits unintended retraction of the distal end portion 112, and corresponding loss of access to the perforated site on the atrial septum 75, during such withdrawal.

The transseptal access system 50 may be configured to achieve a plurality of different curvatures. This is useful to allow introduction into and positioning of the system 50 at a desired location within the heart 20. For example, the various curvatures allow for achieving desired positioning of the dilator 105 and the RF perforation device 110 along a portion of the atrial septum 75.

In some aspects, it may be desirable for the dilator 105, sheath 100, or RF perforation device 110 to include one or more surface electrode. The one or more surface electrode may be located on a distal portion of the dilator 105, sheath 100, or RF perforation device 110 for use in ablation, mapping, pacing, or sensing a parameter within a portion of the heart 20. The one or more surface electrode may be connected to an electroanatomical mapping (EAM) system, generator, or other diagnostic system.

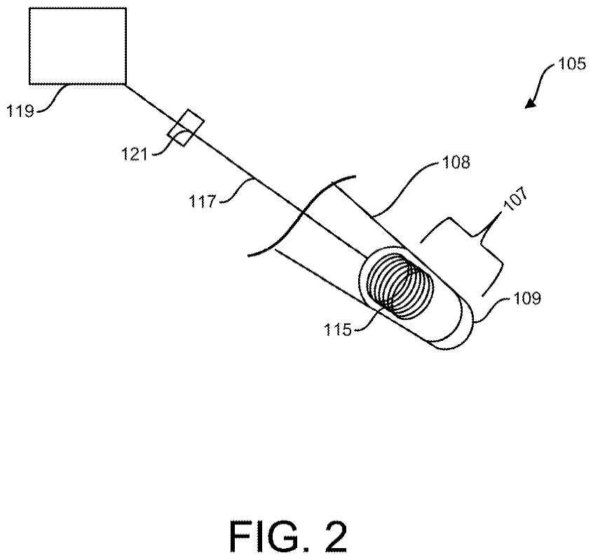

FIG. 2 illustrates a distal portion of a dilator 105 in accordance with an embodiment of the present disclosure. The dilator 105 may be an EAM dilator having one or more electrode 115 positioned on a portion of the dilator 105. The electrode 115 can be configured as a surface electrode that is capable of contacting tissue or fluid within a patient's heart 20. As shown in FIG. 2, the electrode 115 is configured as a coiled electrode 115 positioned along the distal tapered portion 108.

The coiled electrode 115 is formed from a single piece of conductive material that extends from a connector 121 through the dilator 105 and forms the coiled electrode 115. The connector 121 may be removably attached to a system 119. System 119 may include one or more sensors and may be an electroanatomical mapping (EAM) system or a generator capable of generating RF or electricity for the purpose of ablation or puncturing tissue.

The coiled electrode 115 is formed of conductive wire that is looped or coiled at a desired location along the dilator 105. Forming the electrode 115 as a single piece of conductive material is beneficial where wall thickness is important as it allows form a weld-less connection. The coil forming the electrode 115 may have inductive properties which can be taken advantage of for the purposes of localization with an EAM system.

The dilator 105 includes a distal edge 109 that forms a blunt atraumatic tip. Extending proximally of the distal edge 109 is a window 107 that exposes the coiled electrode 115. The distance from the distal edge 109 to the electrode may vary based on a desired use. In some aspects, the window 107 may be positioned only over a portion of the coiled electrode 115. In some aspects, the window 107 may include a feature that allows for adjustment of the size of the window opening. In some aspects, the dilator 109 may include a stiffening member such as a braid or coil. In some aspects, only portions of the dilator109 may include a stiffening member, such as only the proximal portion.

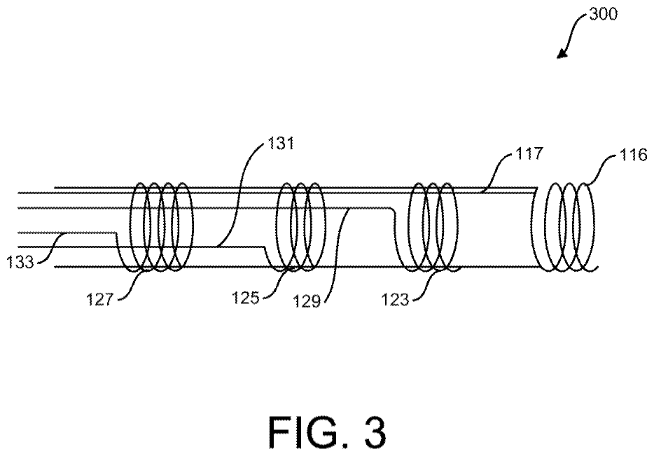

FIG. 3 illustrates a medical device 300 in accordance with an embodiment of the present disclosure. The medical device 300 may be a dilator, perforation device, catheter, guidewire, or other elongate medical device for introduction into a patient.

The medical device 300 includes a tip electrode 116, a distal surface electrode 123, a middle surface electrode 125, and a proximal surface electrode 127. The tip electrode 116 is configured to be used as a puncturing electrode, for use in ablating tissue, or for use in mapping or sensing applications. The distal surface electrode 123, middle surface electrode 125, and proximal surface electrode 127 are configured for use in ablating tissue or mapping and sensing applications. Each of the tip electrode 116, distal surface electrode 123, middle surface electrode 125, and proximal surface electrode 127 have a diameter substantially equal to the surface of the medical device 300.

Each of the tip electrode 116, distal surface electrode 123, middle surface electrode 125, and proximal surface electrode 127 are formed separately. Each of the tip electrode 116, distal surface electrode 123, middle surface electrode 125, and proximal surface electrode 127 can be formed by wrapping a continuous piece of conductive material around an axis of the medical device 300. The tip electrode 116 includes a conductor portion 117 that extends to a proximal end of the medical device. The distal surface electrode 123 includes a conductor portion 129 that extends to a proximal end of the medical device. The middle surface electrode 125 includes a conductor portion 131 that extends to a proximal end of the medical device. The proximal surface electrode 127 includes a conductor portion 133 that extends to a proximal end of the medical device.

In some applications the conductive material is wound around a template, such as a mandrel, prior to positioning on the medical device 300. During assembly, a proximal conductor portion of each of the continuous pieces of conductive material may be threaded though the medical device 300 to a proximal end of the medical device for coupling to a connector 121 or to a system 119.

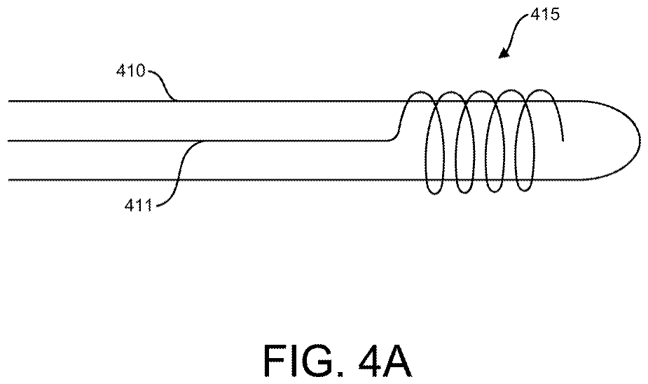

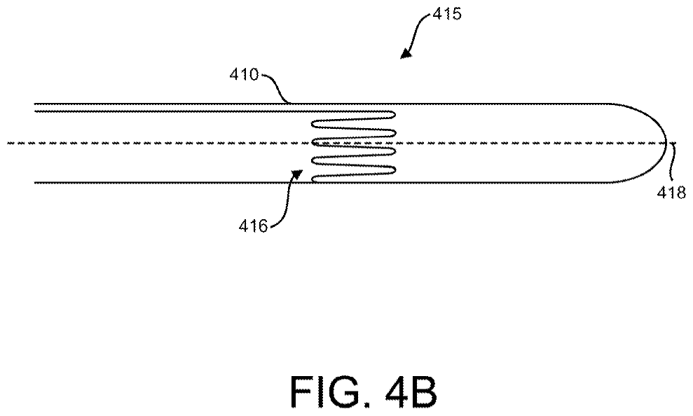

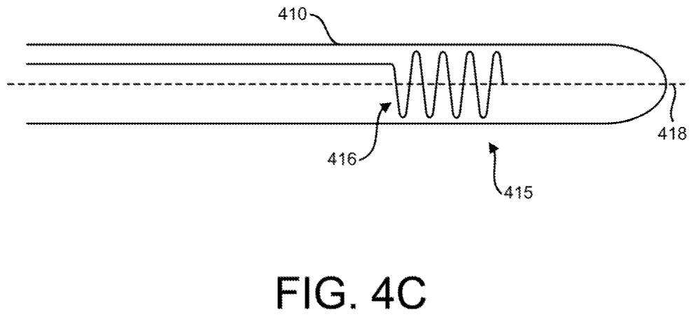

FIGS. 4A-4C illustrate various configurations for forming a coiled electrode 415 in accordance with the present disclosure. As illustrated in FIG. 4A, the coiled electrode 415 may be formed by a single, continuous piece of conductive material wrapped or placed around a medical device 410. For the purposes of use in with an EAM system, the wire can be secured at the most distal end and wrapped around a mandrel, hypotube, or other template to form its coiled shape prior to assembly. The coils should not overlap and lay flat against the template, with minimal space in between each loop. Additional features may be added to secure and debur a distal end of the continuous wire to the coil and add strain relief to the portion of the wire transitioning between the coil electrode 415 and the proximal conductor portion 411. This may be done with solder or an insulating material.

The continuous piece of conductive material may be insulated along the entirety of its length except for the coiled region, which should not be insulated. In some aspects, the loops may be soldered or welded together.

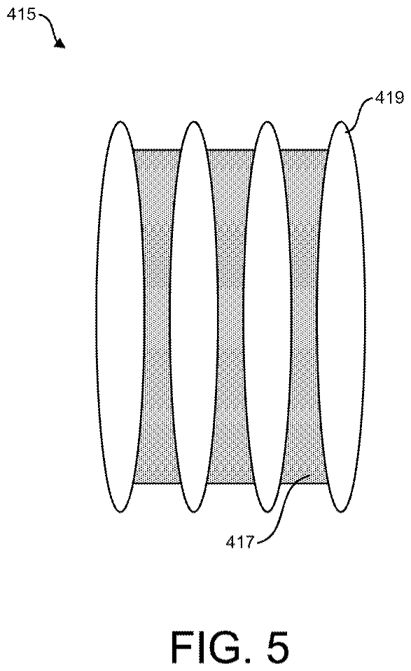

FIG. 5 illustrates an embodiment for joining coils of a coiled electrode in accordance with the present disclosure. As illustrated in FIG. 5, a conductive material 417 may join the loops 419 of the coiled electrode 415. The conductive material 417 may be a solder or a welding material. In some aspects, the conductive material 417 may be the same as material forming the loops 419. In other aspects, the conductive material 417 may be a material different than that forming the loops 419.

FIG. 4B illustrates an embodiment of an electrode 415 formed from a continuous piece of conductive material having a plurality of undulations 416. The plurality of undulations 416 may be arranged in a snake like fashion and then the segments may be electrically and physically connected in a circumferential direction using a conductive material. In FIG. 4B the plurality of undulations are arranged parallel to a longitudinal axis 418 of the medial device 410.

FIG. 4C illustrates another embodiment where the electrode is formed from a continuous piece of conductive material having a plurality of undulations 416. In FIG. 4C, the plurality of undulations are arranged perpendicular to a longitudinal axis 418 of the medical device 410.

FIG. 6 illustrates a medical device 600 in accordance with an embodiment of the present disclosure. The medical device 600 may be a dilator, perforation device, catheter, guidewire, or other elongate medical device for introduction into a patient. The medical device 600 includes an elongate body 601 having a proximal portion (not shown) and a distal portion 603. The elongate body 601 includes a longitudinal axis 618.

The medical device 600 includes a tip electrode 605, a distal surface electrode 607, and a proximal surface electrode 609. The tip electrode 605 is configured to be used as a puncturing electrode, for use in ablating tissue, or for use in mapping or sensing applications. The distal surface electrode 607 and the proximal surface electrode 609 are configured for use in ablating tissue or mapping and sensing applications.

As illustrated in FIG. 6, each of the electrodes 605, 607, 609 have different configurations and sizes, yet each one is formed by a single piece of conductive material. The tip electrode 605 has a diameter that is larger than the body 601 and each of the distal surface electrode 607 and proximal surface electrode 609. The tip electrode 605 is formed by wrapping around a template as described above in FIG. 4A. A proximal conductor portion 619 may extend along an outer surface of the medical device 600.

The distal surface electrode 607 and the proximal surface electrode 609 have a diameter that is approximately equal to the surface of the body 601. Either electrode may be configured to sit slightly above or below the surface of the body 601 if desired for a particular application. While distal surface electrode 607 and proximal surface electrode 609 share a similar diameter, their lengths and configurations are different. A proximal conductor portion of the distal surface electrode 607 and the proximal surface electrode 609 may extend along a lumen within the medical device 600 or within the wall of the medical device 600.

Proximal surface electrode 609 is shown having a length that is greater than the distal surface electrode 607. In some aspects, the distal surface electrode 607 has a length greater than the proximal surface electrode 609. The proximal surface electrode 609 is formed with a plurality of undulations that are parallel to the longitudinal axis 618 as described above in FIG. 4B. The distal surface electrode 607 is formed with a plurality of undulations that are perpendicular to the longitudinal axis 618 as described above in FIG. 4C.

Any of the electrodes shown in FIG. 6 may be connected by a conductive material between adjacent loops or undulations as illustrated and described in FIG. 5.

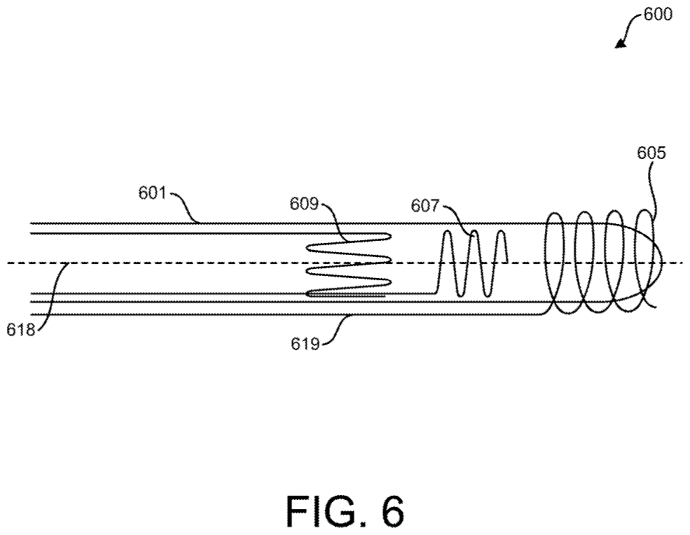

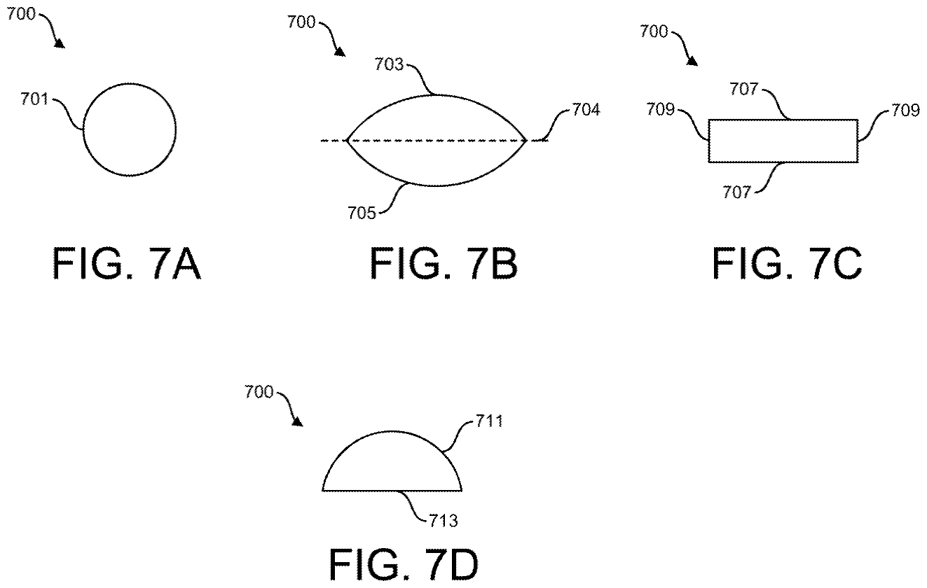

FIGS. 7A-7D illustrate examples of cross-sections of continuous wires that can be used to create any of the coiled electrodes described above. It is understood that FIGS. 7A-7D are illustrative and not exclusive. For example, the wire may include other polygonal or non-polygonal shapes not shown. FIG. 7A illustrates the wire 700 as having a circular cross-section that includes a surface 701 that consists of points equidistant from the center.

FIG. 7B illustrates the wire 700 as having a cross-section having a first curved surface 703 and a second curved surface 705. The first surface 703 and the second curved surface 705 are mirror images over an axis 704 dividing the wire 700.

FIG. 7C illustrates the wire 700 having a rectangular cross-section. The wire includes a first set of parallel surfaces 707 and a second set of parallel surfaces 709 that are positioned orthogonal to the first set of parallel surfaces 707. In some aspects, the first set of parallel surfaces 707 and the second set of parallel surfaces 709 may not be orthogonal to one another, yet remaining at an angle to one another.

FIG. 7D illustrates another example for a cross-section for the wire 700 forming a coiled electrode. In FIG. 7D, the wire 700 includes a first curved surface 711 and a second straight surface 713. In some aspects, the cross-section resembles a dome.

In some aspects, a continuous wire forming a coiled electrode may include a plurality of cross-sectional shapes. For example, a portion of the wire for forming the coiled electrode may have a flat or ribbon shape, while the portion extending from the coiled electrode to the system 119 may include a round or circular shape.

In some aspects, the system 50 can be packaged as a kit and be ready for use right out of the package. The kit may include one or more sheath 100, dilator 105, and RF perforation or puncturing device 110 having one or more coiled electrodes thereon. Alternatively, the kit may include a plurality of dilators 105 each including one or more coiled electrodes and having different pre-shaped portions for use in various procedures.

In some aspects, any of the sheath 100, dilator 105, or RF perforation or puncturing device 110 may include one or more markers along a portion thereof for identifying a position or location during use.

In some aspects, any of the sheath 100, dilator 105, or RF perforation or puncturing device 110 may include a plurality of cuts machined into the wall, for example by laser cutting. The shape and positioning of the cuts can allow for a transition in flexibility from a proximal portion to distal portion. The cuts may include a broken spiral configuration or may be positioned substantially orthogonal to a longitudinal axis of the sheath 100, dilator 105, or RF perforation or puncturing device 110. In some aspects, there may be a single cut that winds around an axis with a wider spacing between loops at the proximal portion and a larger spacing at the distal portion. The spacing and size of the cuts can be varied to achieve different flexibilities along the length of the sheath 100, dilator 105, or RF perforation or puncturing device 110.

In some aspects, any of the sheath 100, dilator 105, or RF perforation or puncturing device 110 may be formed of a shape memory material, such as a shape memory polymer or a shape memory metal. This would allow the sheath 100, dilator 105, or RF perforation or puncturing device 110 to have a first shape at a first temperature, and a second shape at a second temperature. Shape transition may be initiated in the by inserting a heated solution into the sheath 100 or dilator 105 or using electricity to heat a portion of the sheath 100, dilator 105, or RF perforation or puncturing device 110.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

I claim:

1. A medical device, comprising:
an elongate body having a proximal portion, a distal portion, and a lumen extending from the proximal portion to the distal portion;
an electrode located on the distal portion; and
a conductor extending from the electrode to the proximal portion, wherein the electrode and the conductor are formed of a single piece of continuous material.

2. The system of claim 1, wherein the medical device is a dilator, guidewire, catheter, or perforation device.

3. The medical device of claim 1, further comprising:
a first surface electrode positioned proximal of the electrode.

4. The medical device of claim 3, further comprising:
a second surface electrode positioned proximal of the first surface electrode.

5. The medical device of claim 4, wherein the electrode, the first surface electrode, and the second surface electrode have a same diameter.

6. The medical device of claim 4, wherein the first surface electrode and the second surface electrode have different lengths.

7. The medical device of claim 1, wherein the electrode comprises a plurality of loops positioned along a longitudinal axis of the elongate body.

8. The medical device 1, wherein the electrode includes a plurality of undulations.

9. The medical device of claim 8, wherein the plurality of undulations are arranged parallel or perpendicular to a longitudinal axis of the elongated body.

10. The medical device 7, wherein the plurality of loops are joined by a conductive material.

11. The medical device of claim 1, wherein the conductor extends within the lumen to the proximal end.

12. The medical device of claim 1, wherein the conductor is encapsulated in a wall of the elongate body.

13. The medical device of claim 1, wherein the conductor extends along a surface of the elongate body.

14. The medical device of claim 1, wherein the distal portion includes a taper.

15. A medical device, comprising:
an elongate body having a proximal portion and a tapered distal end;
a coiled electrode located on the tapered distal end; and
a conductor extending from the coiled electrode to the proximal portion, wherein the coiled electrode and the conductor are formed of a single piece of continuous material.

16. The medical device of claim 15, further comprising:
a first surface electrode positioned proximal of the coiled electrode.

17. The medical device of claim 16, further comprising:
a second surface electrode positioned proximal of the first surface electrode.

18. The medical device of claim 17, wherein the coiled electrode, the first surface electrode, and the second surface electrode have different lengths.

19. The medical device of claim 15, wherein the coiled electrode comprises a plurality of loops positioned along a longitudinal axis of the elongate body.

20. A method for fabricating a medical device, comprising:
forming a coiled electrode by wrapping a continuous wire around a template for a plurality of turns;
placing the coiled electrode on a distal portion of the medical device; and
threading a proximal portion of the continuous zwire though the medical device to a proximal end of the medical device.
